(19) United States
(12) Patent Application Publication
(10) Pub. No.: US 2016/0290903 A1
(43) Pub. Date: Oct. 6, 2016

(54) CELL-FREE IN VITRO MODELS FOR TRAUMATIC BRAIN INJURY AND METHODS FOR PREPARATION AND USE THEREOF

(71) Applicant: President and Fellows of Harvard College, Cambridge, MA (US)

(72) Inventors: Kevin Kit Parker, Cambridge, MA (US); Leila F. Deravi, Durham, NH (US); Borna E. Dabiri, Cambridge, MA (US)

(21) Appl. No.: 15/034,901

(22) PCT Filed: Nov. 7, 2014
(86) PCT No.: PCT/US14/64590

§ 371 Date: May 6, 2016

Related U.S. Application Data
(60) Provisional application No. 61/901,087, filed on Nov. 7, 2013.

(51) Int. Cl. G01N 3/08; C08L 89/00; C08L 25/18; C07K 14/78

(52) U.S. Cl. CPC G01N 3/08; C07K 14/78; C08L 89/00; C08L 25/18

(57) ABSTRACT

The present invention provides in vitro models of non-penetrating traumatic brain injury, which include a network of extracellular matrix (ECM) protein fibrils, methods for preparing such models, and uses of such models for, e.g., identifying compounds suitable for preventing or treating non-penetrating traumatic brain injury.

### RELATED APPLICATIONS

This application claims priority to U.S. Provisional Application No. 61/901,087, filed on Nov. 7, 2013, the entire contents of which are incorporated herein by this reference.

### GOVERNMENT SUPPORT

This invention was made with government support under W81XWH-11-2-0057 awarded by the U.S. Department of Defense (DARPA). The United States government has certain rights in the invention.

### BACKGROUND OF THE INVENTION

Traumatic brain injury (TBI) occurs when an external mechanical force causes brain dysfunction. Traumatic brain injury usually results from a violent blow or jolt to the head or body. The severity of a TBI may range from “mild” to “severe.” An object penetrating the skull, such as a bullet or shattered piece of skull, also can cause traumatic brain injury. A mild traumatic brain injury, such as a non-penetrating traumatic brain injury, may cause temporary dysfunction of brain cells. Severe traumatic brain injury can result in bruising, torn tissues, bleeding and other physical damage to the brain that can result in long-term complications or death.

In a traumatic brain injury (TBI), mechanical forces applied to the central nervous system (CNS) disrupt the extracellular matrix (ECM) by direct force or activation of specific enzymatic pathways. In the central nervous system (CNS), the extracellular matrix (ECM) links neuronal, glial and vascular compartments together through specific ligand-receptor interactions. The ECM is composed of integrins, cell adhesion molecules, and glycoproteins, which are constantly remodeled to promote CNS functions. As a result of disease or injury, the ECM is susceptible to damage, alterations and modifications.

According to the Centers for Disease Control and Prevention, each year, traumatic brain injuries contribute to a substantial number of deaths and cases of permanent disability.

For example, in the four year time period between 2002-2006, the estimated average annual number of traumatic brain injury cases in United States alone was 1.7 million, which included 52,000 deaths, 275,000 hospitalizations, 1.365 million (about 80% of the total) emergency department visits. (www.cdc.gov/traumaticbraininjury/pdf/Bluebook_factsheet-a.pdf). Notably, about 75% of traumatic brain injuries that occur each year are concussions or other forms of mild traumatic brain injury (e.g., due to a fall).

Accordingly, there is a need in the art for methods and compositions for preventing or treating traumatic brain injury, particularly mild or non-penetrating traumatic brain injury, as well as compositions and methods for identifying compounds suitable for preventing or treating non-penetrating traumatic brain injury.

### SUMMARY OF THE INVENTION

The present invention provides compositions and methods for identifying compounds suitable for preventing or treating non-penetrating traumatic brain injury. The present invention is based, at least in part, on the development of an in vitro model of non-penetrating traumatic brain injury. Without intending to be limited by any theory or mechanism of action, the in vitro model relies on the fact that large strains on the extracellular matrix (ECM) protein used in the model for protein fibril network formation, can cause domain unfolding at the molecular level and plastic deformation at the fibril level, and that these changes can be detected using a suitable method.

Accordingly, in one aspect, the present invention provides a cell-free in vitro model of non-penetrating traumatic brain injury including a base layer including a stretchable membrane disposed on a surface, a network of extracellular matrix (ECM) protein fibrils on the stretchable membrane structured to mimic the brain perineuronal network, such that the network of ECM protein fibrils includes a plurality of nodes, such that each node includes a polymer having a charge density of about 0.10 C/m² or greater, such that each node is independently spaced from each of its immediate neighbors by about 20 μm to about 40 μm, such that each fibril is attached to one or more of the plurality of nodes, and each fibril intersects another fibril at a node, such that the stretchable membrane comprising the plurality of nodes and the network of fibrils is stretched, thereby mimicking non-penetrating traumatic brain injury.

In another aspect, the invention provides a cell-free in vitro model of non-penetrating traumatic brain injury, including a base layer including a stretchable membrane disposed on a surface, a network of extracellular matrix (ECM) protein fibrils on the stretchable membrane structured to mimic the brain perineuronal network, such that the network of ECM protein fibrils includes a plurality of nodes, such that each node includes a polymer having a charge density of about 0.10 C/m² or greater, such that each node is independently spaced from each of its immediate neighbors by about 20 μm to about 40 μm, such that each fibril is attached to one or more of the plurality of nodes, and each fibril intersects another fibril at a node, such that the stretchable membrane comprising the plurality of nodes and the network of fibrils when stretched, mimics non-penetrating traumatic brain injury.

In one embodiment, each fibril is attached to at least one node and at least one of its nearest neighbor nodes.

In another embodiment, the network of extracellular matrix proteins includes a network of fibrils selected from the group consisting of aggrecan fibrils, brevican fibrils, neurocan fibrils, tenascin R fibrils, and any combination thereof.

In one embodiment, the network of extracellular matrix proteins includes a network of aggrecan fibrils.

In another embodiment, the network of extracellular matrix proteins includes a network of brevican fibrils.

In yet another embodiment, the network of extracellular matrix proteins includes a network of neurocan fibrils.

In a further embodiment, the network of extracellular matrix proteins includes a network of tenascin R fibrils

In one embodiment, the base layer has an elasticity of about 0.5 megapascal (MPa) to about 1.5 MPa, about 0.75 MPa to about 1.25 MPa, about 1.0 MPa to about 1.5 MPa, about 0.5 MPa to about 1.0 MPa, about 0.8 MPa to about 1.0 MPa, about 1.0 MPa to about 1.2 MPa, about 1.2 MPa to about 1.4 MPa, or about 1.3 MPa to about 1.5 MPa.

In one embodiment, the network of extracellular matrix proteins is mechanically stretched.

In one embodiment, the base layer comprises a silicone membrane.

In one embodiment, the network of extracellular matrix proteins is stretched at a strain rate of about 0.1 to 10% per millisecond (msec⁻¹), about 1.0 to 9% msec⁻¹, about 2.0 to 8% msec⁻¹, about 3.0 to 7% msec , about 4.0 to 6% msec⁻¹, about 0.1 to 1.0% msec⁻¹, about 1.0 to 2.0% msec⁻¹, about 2.0 to 3.0% msec⁻¹, about 3.0 to 4.0% msec⁻¹, about 4.0 to 5.0% msec⁻¹, about 5.0 to 6% msec⁻¹, about 6.0 to 7.0% msec⁻¹, about 7.0 to 8.0% msec⁻¹, about 8.0 to 9.0% msec⁻¹, or about 9.0 to 10.0% msec⁻¹.

In one embodiment, the network of extracellular matrix proteins is stretched at a displacement rate of about 25 μmsec⁻1 to about 1000 μm sec⁻¹, about 100 μm sec⁻1 to about 1000 μm sec⁻¹, about 200 μm sec⁻1 to about 900 μm sec⁻¹, about 300 μm sec⁻1 to about 800 μm sec⁻¹, about 400 μm sec⁻1 to about 700 μm sec⁻¹, about 500 μm sec⁻1 to about 600 μm sec⁻¹, about 25 μm sec⁻1 to about 100 μm sec⁻¹, about 100 μm sec⁻1 to about 200 μm sec⁻¹, about 200 μm sec⁻1 to about 300 μm sec⁻¹, about 300 μm sec⁻1 to about 400 μm sec⁻¹, about 400 μm sec⁻1 to about 500 μm sec⁻¹, about 500 μm sec⁻1 to about 600 μm sec⁻¹, about 600 μm sec⁻1 to about 700 μm sec⁻¹, about 700 μm sec⁻1 to about 800 μm sec⁻¹, about 800 μm sec⁻1 to about 900 μm sec⁻¹, or about 900 μm sec⁻1 to about 1000 μm sec⁻¹.

In one embodiment, the network of extracellular matrix proteins is stretched at a strain rate of about 1% msec⁻¹ and a displacement rate of about 500 μm sec⁻¹.

In one embodiment, the polymer has a charge density of about 0.01 C/m² to about 10 C/m², about 0.1 C/m² to about 10 C/m², about 1.0 C/m² to about 10 C/m², about 5.0 C/m² to about 10 C/m², about 0.01 C/m² to about 5 C/m², about 0.01 C/m² to about 1.0 C/m², about 0.01 C/m² to about 0.1 C/m², about 0.1 C/m² to about 1.0 C/m², about 0.5 C/m² to about 1.0 C/m², about 0.1 C/m² to about 0.2 C/m², about 0.05 C/m² to about 0.1 C/m², about 0.15 C/m² to about 0.2 C/m², or about 0.2 C/m² to about 0.3 C/m².

In one embodiment, the polymer is a polystyrene sulfonate.

In one embodiment, the polymer is poly(styrene-co-4-styrene sulfonic acid).

In another aspect, the present invention provides a method for identifying a compound useful for preventing or treating a non-penetrating traumatic brain injury, the method including providing a cell-free in vitro model of traumatic brain injury of the invention; contacting the cell-free in vitro model with a test compound and examining the structure of the network of extracellular matrix (ECM) protein fibrils of the model; generating a mechanical strain in the network of ECM protein fibrils previously determined to be sufficient to cause a structural change in the network; examining the structure of the network after the generation of the mechanical strain for the presence of a change; and identifying the test compound as a compound useful for preventing or treating a traumatic brain injury if the change in the structure of the network after the generation of the strain is not substantial or is absent.

In one embodiment, the change in the network is measured quantitatively.

In one embodiment, the change in the network is a change in the geometric structure of the network.

### BRIEF DESCRIPTION OF THE DRAWINGS

FIG. 1 depicts the process (top) of forming a network of extracellular matrix protein fibrils as part of a cell-free in vitro model of non-penetrating traumatic brain injury. Nodes (dark circles) containing Poly(styrene-co-4-styrene sulfonic acid) (PSS) were micro patterned on a base layer. The patterned base layer was incubated with an ECM protein solution. Further incubation with polyvinyl alcohol resulted in the generation of anisotropic networks (bottom; light color).

FIG. 2 shows network formation using a number of different proteins and chondroitin sulfate proteoglycans (CSPGs).

FIG. 3 shows an apparatus for high-speed stretching of protein networks for simulating non-penetrating traumatic brain injury. Protein networks were formed on stretchable silicone membranes. Membranes subjected to mechanical strain above a certain threshold suffer a loss in structural integrity, as shown, for example, by a change in the geometry of the network.

FIGS. 4A and 4B show fluorescent images for the characterization of mild traumatic brain injury using the cell-free in vitro model with protein fibril networks from FIG. 1. In

FIG. 4A, confocal fluorescence microscopy images of a protein fibril network before and after an injury (stretch) are shown. Arrows point to visible changes in network anisotropy due to injury. FIG. 4B shows a fluorescence image of a protein network before injury (dark) overlaid on the network after injury(light).

FIGS. 5A, 5B and 5C show the process of obtaining network cross-correlation between the states of the network before and after a stretch. The vertical shadow in FIG. 5A going through the top, the middle, and the bottom panels indicates pixels in the fluorescent images of the network (such as in FIG. 4) that are used for obtaining the cross-correlation. FIG. 5B represents a control (sham) in which the network was not subjected to a stretch, and therefore image cross-correlation was perfect (value of 1). FIG. 5C is a graph showing image cross-correlation in a region of interest (ROI) in a stretched (injured) membrane relative to cross-correlation in an unstretched membrane (sham; no injury).

FIGS. 6A, 6B and 6C show network cross-correlation across a larger region (500 μm²) compared to that in FIG. 5 (100 μm²). The static images (A, B) are captured before and after 100% strain using confocal microscopy and analyzed using custom MatLab script. Images were cross-correlated to elucidate changes in network structure.

### DETAILED DESCRIPTION OF THE INVENTION

The present invention provides a cell-free in vitro model of non-penetrating traumatic brain injury or a closed-head injury such as those caused by vehicular accidents, falls, acts of violence, or sports injuries. The invention further provides a method of identifying a compound for treating or preventing a non-penetrating traumatic brain injury, using the cell-free in vitro models of the invention.

### I. An In Vitro Model of Non-Penetrating Traumatic Brain Injury

In one aspect, the present invention provides a cell-free in vitro model of non-penetrating traumatic brain injury that includes a base layer having a stretchable membrane disposed on a surface and a network of extracellular matrix (ECM) protein fibrils on the stretchable membrane structured to mimic the brain perineuronal network. The ECM protein fibril network includes a plurality of nodes. The nodes include a polymer having a charge density of about 0.10 C/m² or greater. Each node is independently spaced from its immediate neighbors by a distance of about 20 μm to about 40 μm. Each extracellular matrix protein fibril is attached to one or more of nodes, and each fibril intersects another fibril at a node. The base layer, which includes the nodes and fibrils, is stretched to mimic a non-penetrating traumatic brain injury.

As used herein, “non-penetrating traumatic brain injury” or “closed head injury” is a type of traumatic brain injury in which the skull and dura mater remain intact. Closed-head injuries are caused primarily by vehicular accidents, falls, acts of violence, and sports injuries. In addition, blast-related traumatic brain injuries are often closed-head injuries and result from rapid changes in atmospheric pressure, objects dislodged by the blast hitting people, or people being thrown into motion by the blast.

As used herein, the term “node” is a point on a base layer at which two extracellular matrix fibrils intersect.

As used herein, the term “stretchable membrane” refers to a membrane that can be stretched. A membrane is stretchable when it can be extended in any direction it is pulled in. An example of a stretchable membrane is a polydimethylsiloxane (PDMS) membrane. A flexible membrane can also be a stretchable membrane if it can be extended to 2, 3, 4, 5, 6, 7, 8, 9, or 10 times its original dimension in the direction of the pull.

As used herein, “charge density” is the amount of electric charge per unit surface area (Coulombs (C)/m²). The polymer used in the in vitro models of the present invention may have a charge density of, for example, about 0.01 C/m² to about 10 C/m², about 0.1 C/m² to about 10 C/m², about 1.0 C/m² to about 10 C/m², about 5.0 C/m² to about 10 C/m², about 0.01 C/m² to about 5 C/m², about 0.01 C/m² to about 1.0 C/m², about 0.01 C/m² to about 0.1 C/m², about 0.1 C/m² to about 1.0 C/m², or about 0.5 C/m² to about 1.0 C/m².

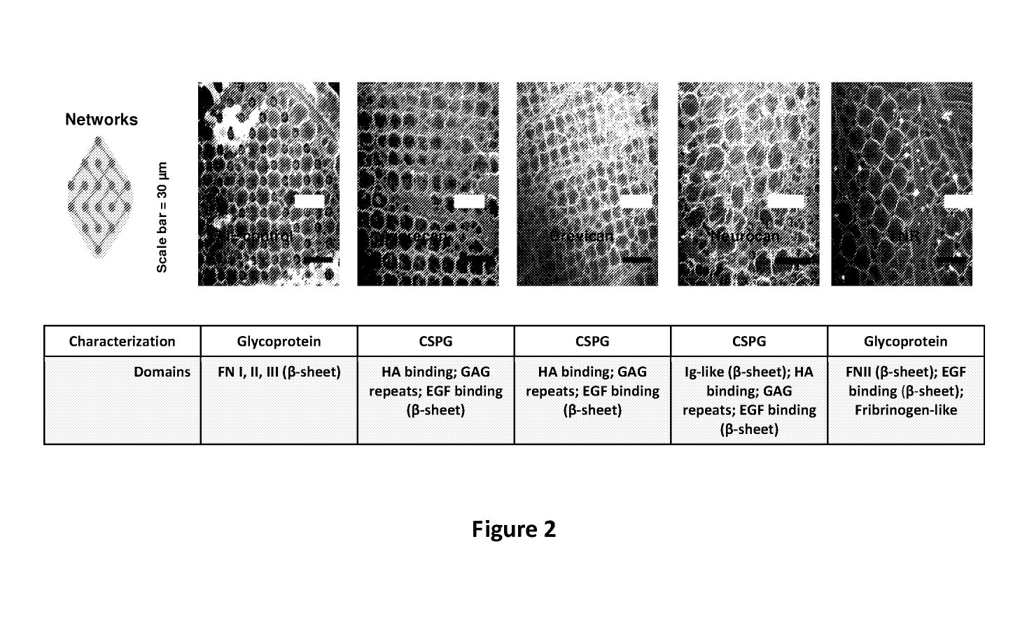

The model of the present invention comprises a network of extracellular matrix (ECM) protein fibrils formed on a base layer. The network of ECM protein fibrils is formed so as to mimic the brain perineuronal network. The perineuronal net (PNN) is a stabilizing structure in the brain with partial negative charges and includes net negatively charged molecules such as (a) chondroitin sulfate proteoglycans (CSPGs; e.g., aggregan, brevican, and neurocan, (b) glycoproteins (e.g., tenascin R (TN-R)), and (c) glycosaminoglycans (GAGs; e.g., hyaluronic acid (HA)) (FIG. 2).

The ECM protein fibril network includes a plurality of nodes. Each node comprises a polymer having a charge density equal to or greater than the charge of the surface of a cell (e.g., 0.10 C/m²). Each node is independently spaced from its nearest neighbor by about 20 μm to about 40 μm, e.g., about 20 μm to about 30 μm, about 30 μm to about 40 μm, about 20 μm to about 25 μm, about 25 μm to about 30 μm, about 30 μm to about 35 μm, or about 35 μm to about 40 μm. Each ECM protein fibril is attached to one or more nodes, and intersects with another fibril at a node to generate the network. Stretching the flexible (also stretchable) membrane on the base layer stretches the network of protein fibrils, thereby mimicking the perineuronal network of a brain that has suffered a non-penetrating traumatic brain injury.

A number of different ECM proteins may be used for generating protein fibril network for the in vitro model. Examples of such proteins include chondroitin sulfate proteoglycans (CSPGs), such as aggregan, brevican, and neurocan; glycoproteins, such as tenascin R (TN-R); and glycosaminoglycans (GAGs), such as hyaluronic acid (HA). Without intending to be limited by any theory, it is believed that network formation is dependent on the presence of beta-sheet domains within the proteins.

A. ECM Protein Fibril Network Formation

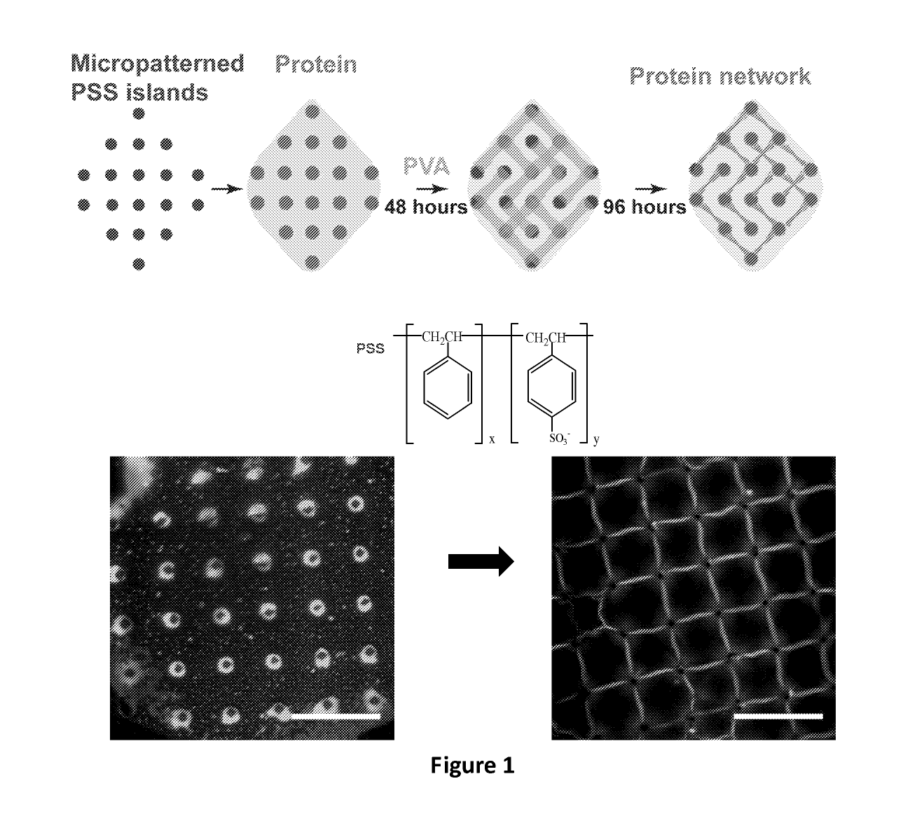

Formation of a network of extracellular matrix protein fibrils according to the present invention is generally depicted in FIG. 1. A polymer with a charge density equivalent to or higher than the charge on mammalian cell surfaces in situ (e.g., about 0.01 C/m² to about 10 C/m²) is applied at specific locations on a stretchable membrane on a base layer to form a plurality of nodes resulting in a micro pattern of nodes. A suitable polymer for node formation is any negatively charged polymer with a charge density that mimics the charge density of the plasma membrane of the cell. Suitable polymers for use as nodes in the invention facilitate the self-assembly and self-organization of brain extracellular matrix proteins into a network by initiating unfolding of fibrillar extracellular matrix proteins and fibrillogenesis of the fibrillar extracellular matrix proteins into fibrils. An example of such polymer is Poly(styrene-co-4-styrene sulfonic acid) (PSS). PSS contains a variable number (16-44 mole %) of sulfonate groups covalently bound to aromatic rings of polystyrene, allowing nodes of different amount of charges to be formed. The charged nodes function to initiate protein unfolding and fibrillogenesis at the nodes to facilitate protein fibril network formation. The minimum charge density that a node should have in order to allow formation of the network is dependent on the protein used for the formation of the network. For example, if the protein used is fibronectin, network formation requires PSS having greater than 16 mol % of sulfonate groups. PSS with 18 mol % sulfonate groups is equivalent to a charge of about 0.12 C/m². The relationship between mol % of sulfonate groups and resulting charge is described in Pernodet et al. Journal of Biomedical Materials Research Part A, 2003, 64, 684, the entire contents of which are incorporated herein by reference.

The polymer is next coated on another material, for example, a wafer of Polydimethylsilane (PDMS; Sylgard 184 (Dow Corning) to produce a stamp for facilitating the delivery of the polymer to the stretchable or flexible (also stretchable) membrane on the base layer. As exemplified and described in greater detail in the Examples section, stamps were produced by coating PDMS wafers with a solution of PSS33 in 50% DMF and 50% PBS. Stamps for generating negative control nodes that are not capable of facilitating protein fibril network formation may be prepared using a PSS polymer of lower charge such as PSS16. After removal of the excess coating solution by a flow of air, the stamps are contacted with the flexible (also stretchable) membrane of the base layer for about an hour. The patterned base layers thus produced are stabilized in an oven at 37° C.

The base layer comprises a stretchable membrane placed on a rigid or semi-rigid material such as a plastic, metal, ceramic, or a combination thereof. Examples of rigid or semi-rigid materials that can be used to form the base layer include polystyrene, polycarbonate, polytetrafluoroethylene (PTFE), polyethylene terephthalate, quartz, silicon, and glass. In one embodiment, the rigid component of the base layer is a silicon wafer, a glass cover slip, a multi-well plate, a tissue culture plate, a Petri dish, or a microfluidic chamber. The base layer is ideally biologically inert.

In one embodiment, the stretchable membrane of the base layer comprises PDMS.

In another embodiment, the stretchable membrane of the base layer comprises PDMS coated on a sacrificial layer comprising Poly(N-isopropylacrylamide) (PIPAAm)

(Polysciences, Inc.). The base layer comprising the stretchable membrane having PDMS coated on PIPAAm was fabricated in a multistep spin coating process as follows. PIPAAm was dissolved at 10 wt % in 99.4% 1-butanol (w/v) and spun coated onto the glass cover slips. PDMS elastomer was mixed at a 10:1 base to curing agent ratio and spin coated on top of the PIPAAm coated glass cover slip. Polydimethylsiloxane-coated cover slips were then cured.

Next, the patterned base layer is incubated in an oven at 37° C. with a solution of an ECM protein conjugated with an agent that binds to the protein and facilitates imaging, e.g., a fluorophore. A fluorophore that may be used for this purpose is, for example, Alexa Fluor 488 carboxylic acid, 2,3,5,6-tetrafluorophenyl ester (Molecular Probes, Invitrogen). After protein incubation, the base layer is washed, and incubated for about 48 hours at 37° C. with a water soluble polymer, such as polyvinyl alcohol (0.5% (v/v)), suitable for production of fibers that can be manipulated (e.g. spun, coagulated, oriented, and cross-linked) under conditions similar to those used for protein fibers. Use of polyvinyl alcohol with a protein for mixed fiber production is described, for example, in Zhang et al. Journal of Applied Polymer Science, 1999, Vol. 71, 11-19, the entire contents of which are incorporated herein by reference.

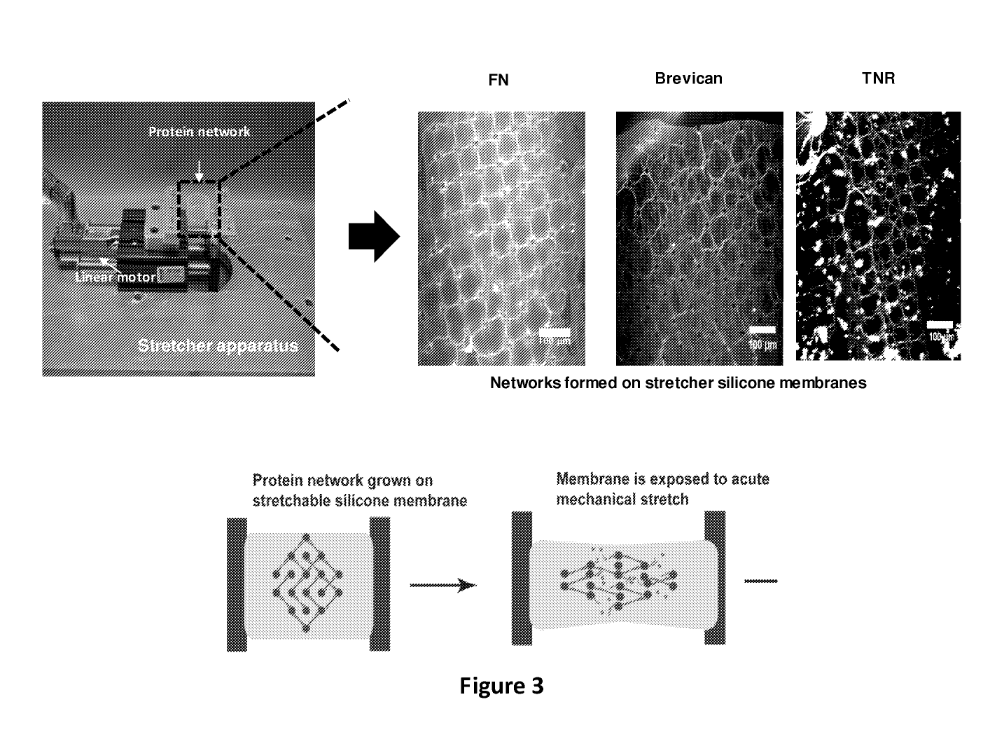

The protein fibril network thus formed may be “injured” by subjecting it to a strain, e.g., mechanical strain. The mechanical strain may be applied using a high-speed stretcher apparatus (FIG. 3). Above a certain threshold, the mechanical strain results in a change in the structural integrity of the protein fibril network. For example, the stretching force applied may be of a magnitude sufficient to generate a displacement rate of about 250 μm sec⁻¹. This displacement rate produces a mechanical strain rate of 1% msec⁻¹.

B. Quantitation of Structural Change Produced Due to Strain

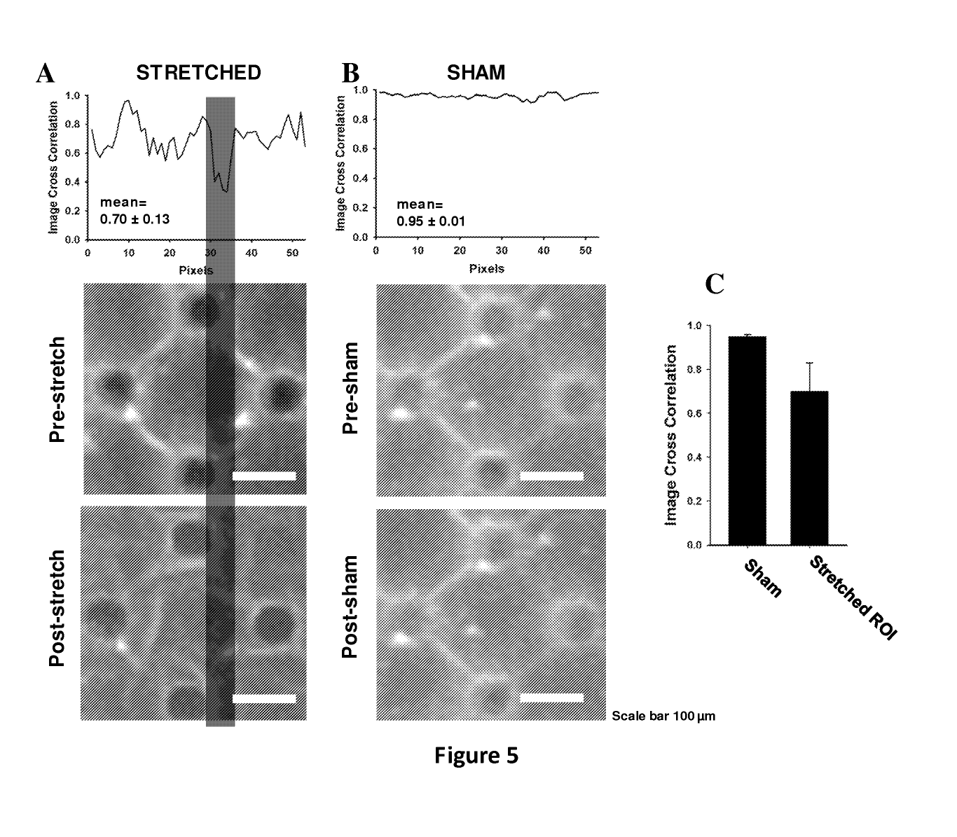

The networks subjected to a strain, e.g., a mechanical strain, may be analyzed qualitatively or quantitatively for changes to the network. A quantitative measure of the effect of the mechanical strain on the protein fibril network, in accordance with the invention, is obtained through an analysis of pixel intensities of confocal fluorescent images of the network before and after stretch. The analysis, exemplified in the Examples section, involves dividing each image into one pixel wide columns as shown in FIG. 5. Next, pixel intensities for each column of the pre-stretch image is compared or cross-correlated with pixel intensities of the corresponding column of the post-stretch image. A perfect or nearly perfect match is assigned a cross-correlation value of 1, and a lack of a perfect or a nearly perfect match is represented with a cross-correlation value less than 1. An average of the cross-correlation values of columns over the entire image is computed to obtain a net image cross-correlation value. A net image cross-correlation value of 1 indicates that the structural integrity of the network, e.g., the geometry, was maintained in spite of the mechanical strain. A net image cross-correlation value of less than 1 indicates that the structural integrity of the network was compromised or diminished as a result of the applied mechanical strain.

“Cross-correlation” is a standard method of estimating the degree to which two series are correlated. In the in vitro model of the invention, and methods using the in vitro model described herein, cross-correlation refers to correlation of pixel intensities of images (e.g. confocal microscopy images) of a region of a protein fibril network, before and after a stretch is applied. The stretched and unstretched protein fibril networks may be imaged and the image divided into pixel wide columns. Pixel intensities of each column are cross-correlated between pre-stretch (sham) and post-stretch images and the correlations may be averaged over multiple columns to provide a net correlation (mean) of the image. A value of 1 for the cross-correlation indicates that there were no changes to the network.

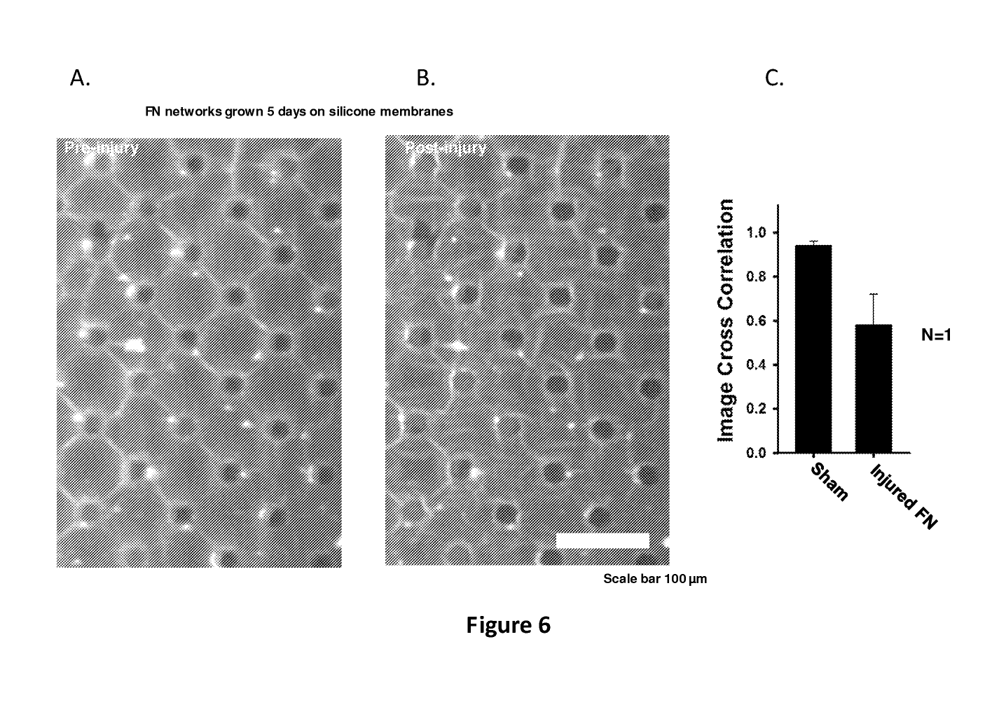

The results of a network cross-correlation analysis performed as described above, in a small region (100 μm²) are shown in FIG. 5. The observed drop in cross-correlation between the intensities of the pixels before and after a stretch in particular regions of the image, as shown in the graph in FIG. 5A, shows the presence of injury in the protein fibril network. Results from another example of applying a stretching force on a fibronectin fibril network generated according to the invention is shown in FIG. 6. In this example, network cross-correlation data was obtained by analyzing pixel intensities over a large region (500 μm²). The static confocal fluorescent microscopy images (FIGS. 6A and 6B) were captured before and after subjecting the network to a strain rate of 100% msec⁻¹. The images were analyzed using custom MatLab script, and cross-correlated to assess changes in the structural integrity of the network (FIG. 6C).

### II. Screening Assays Using the Models of the Invention

In another aspect, the present invention provides methods for identifying a compound useful for preventing or treating a non-penetrating traumatic brain injury. The methods include (a) providing the cell-free in vitro model of traumatic brain injury described above; (b) contacting the cell-free in vitro model with a test compound, and examining the structure of the ECM protein fibrils of the model; (c) generating a mechanical strain in the network of ECM protein fibrils, the strain having previously been determined to be sufficient to cause a structural change in the network; (d) examining the structure of the network of ECM protein fibrils after the generation of the mechanical strain; and (e) identifying the test compound as a compound useful for preventing or treating the non-penetrating traumatic brain injury if (in the presence of the test compound) the structure of the network after the generation of the strain is substantially the same (i.e., no substantial change or no change) as the structure of the network before the generation of the strain.

In one embodiment, the change in the network is a quantitative change.

In one embodiment, the change in the network is a change in the geometric structure of the network.

As used herein, the term “contacting” is intended to include any form of interaction (e.g., direct or indirect interaction) of a test compound and a network of ECM protein fibrils. The term contacting includes incubating a compound and a network of ECM protein fibrils together (e.g., adding the test compound to a network of ECM protein fibrils on a stretchable membrane).

In accordance with the invention, the in vitro model of non-penetrating traumatic brain injury is contacted with a test compound, and optionally incubated for a period of time, for example, 5 minutes to 2 hours. The network is imaged with a confocal fluorescent microscope. A stretching force is next applied to the protein network, and another confocal microscopy image of the network is acquired. Changes in the structure, e.g., geometry, of the network are determined by computing cross-correlation of pixel intensities between the pre-stretch and the post-stretch images. A compound effective in substantially preserving the pre-stretch structure, e.g., geometry, of the protein network, or that results in only a minimal (not substantial) change to the pre-stretch structure, e.g., geometry, i.e., a cross-correlation value of 1 or close to 1, is identified as a compound that is effective for preventing or treating a non-penetrating traumatic brain injury.

Test compounds, may be any agent including chemical agents (such as toxins), small molecules, pharmaceuticals, peptides, proteins (such as antibodies, cytokines, enzymes, and the like), nanoparticles, and nucleic acids, including gene medicines and introduced genes, which may encode therapeutic agents, such as proteins, antisense agents (i.e., nucleic acids comprising a sequence complementary to a target RNA expressed in a target cell type, such as RNAi or siRNA), ribozymes, and the like.

The test compound may be added to the ECM protein fibril network by any suitable means. For example, the test compound may be added drop-wise onto the surface of the stretchable membrane of the base layer of the invention and allowed to diffuse.

In the methods of the invention, a change in the network of the model of traumatic brain injury may be qualitative or quantitative. For example, in some embodiments, visual inspection of the network may reveal changes in geometric structure of the network, e.g., the orientation of the fibrils, or the spacing of the fibrils.

In one embodiment, the changes are physical changes which are quantitative as described herein. For example, a network may be imaged before and after stretching. Appropriate controls may also be imaged. An appropriate control is, for example, a network that is not stretched but maintained under the same conditions as a stretched network.

The stretched networks may be imaged and the image may be divided into columns. Pixel intensities of each column may be cross-correlated between pre-stretch (sham) and post-stretch images and the correlations may be averaged to provide a net correlation (mean) of the image. A value of 1 for the cross-correlation indicates that there were no changes to the network.

This invention is further illustrated by the following examples which should not be construed as limiting. The contents of all references, patents and published patent applications cited throughout this application, as well as the Figures, are hereby incorporated herein in their entirety by reference.

### EXAMPLES

### Example 1

### An In Vitro Model of Non-Penetrating Traumatic Brain Injury

The extracellular matrix (ECM) is the stabilizing structure of the brain. Organized ECM structures have been identified at perineuronal nets, synapses, and Nodes of Ranvier. Changes in ECM protein composition and conformation impact brain function. The perineuronal net (PNN) is a stabilizing structure in the brain. It has a negative charge due to the presence of chondroitin sulfate proteoglycans (CSPGs), such as aggregan, brevican, and neurocan; glycoproteins, such as tenascin R (TN-R); and glycosaminoglycans (GAGs), such as hyaluronic acid (HA) (FIG. 2).

The ECM protein Fibronectin, was used to generate a protein fibril network as part of an in vitro model to mimic non-penetrating traumatic brain injury (FIG. 1). The model relies on the fact that large strains on the ECM protein (e.g., fibronectin) used for the network formation can cause domain unfolding on the molecular level and plastic deformation on the fibril level which can be detected using a suitable method. As a first step, a protein fibril network was formed on a base layer. The base layer comprises a sretchable membrane stabilized by a surface such as tissue culture petri dishes or glass coverslips. To mimic the negative charge in the perineuronal net a negatively charged polymer poly(styrene-co-4-styrene sulfonic acid) (PSS) was used. Specifically, the PSS used is PSS 33, which contains 33 mole % of SO₃H.

The PSS was applied at specific spots on the stretchable membrane of the base layer to form of a pattern of nodes using the process of microcontact printing (FIG. 1). The charged nodes function to initiate protein unfolding and fibrillogenesis to facilitate protein fibril network formation. Microcontact printing was carried out by first fabricating stamps composed of polydimethylsilane (PDMS, Sylgard 184, Dow Corning). These stamps are formed with microscale features templated by silicon wafers pre-patterned using soft lithography. The stamps were coated for 2 hours at room temperature with 20-μg/ml PSS33 dissolved in 50% DMF and 50% PBS solutions prior to deposition. PSS 16 dissolved in 50% DMF and 50% PBS solutions was used to produce stamps for producing negative control nodes that cannot facilitate protein fibril network formation. After the 2 hour incubation, the stamps were dried by removing excess polymer solution by blowing air. The stamps were next placed in contact with the stretchable membrane of the base layer (e.g., a stretchable silicone membrane supported by tissue culture plastic or a glass coverslip). The base layer with the membrane had previously been treated in a UV-ozone cleaner (Jelight Company, Inc.). After a contact period of 1 hour, the patterned base layers were stabilized overnight in an oven at 37° C.

Next, a network of ECM protein was formed on the patterned polymer surfaces (base layer). The patterned base layer was incubated with 1 mL of a 100-μg/ml of the ECM proteins conjugated with Alexa Fluor 488 carboxylic acid, 2,3,5,6-tetrafluorophenyl ester (Molecular Probes, Invitrogen) in PBS for 6 hours in an oven at 37° C. After protein incubation, the base layers were washed and directly incubated with 0.5% (v/v) polyvinyl alcohol (PVA, Mowiol® 4-88, Sigma Aldrich) for 48 hours at 37° C. resulting in the formation of an ECM protein fibril network (FIG. 1).

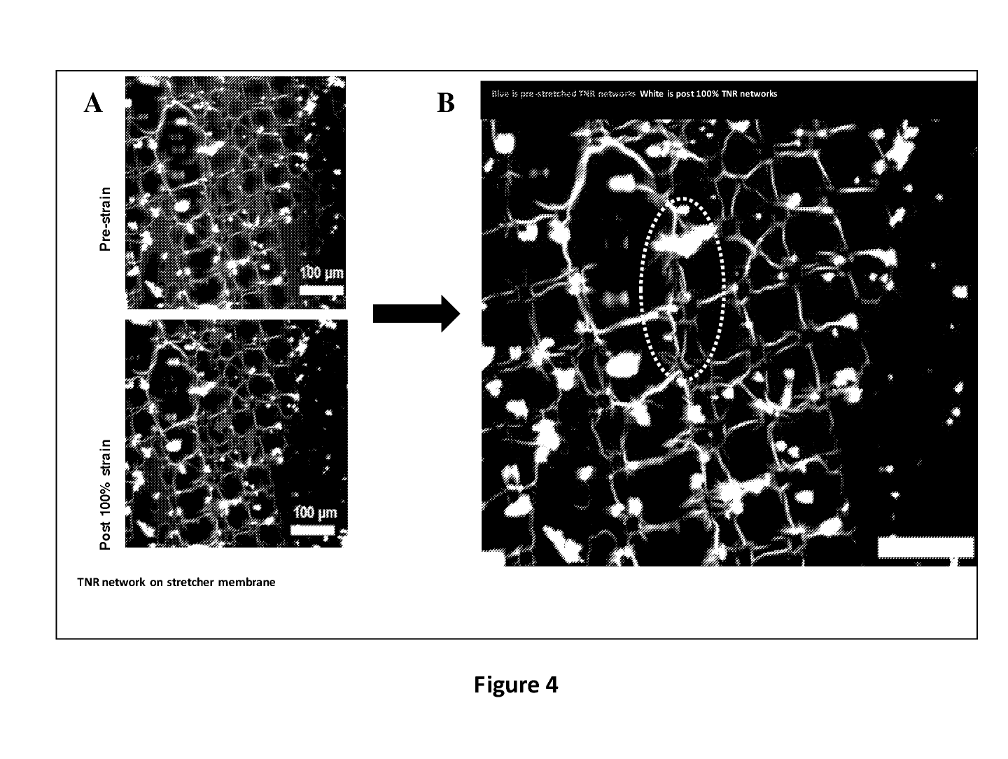

The protein fibril network thus formed was subjected to mechanical strain to mimic a non-penetrating traumatic brain injury by stretching using various amounts of force. The force was applied using a high-speed stretcher apparatus (FIG. 3). Typically, the stretching force applied was of a magnitude sufficient to generate a displacement rate of 250 μm sec⁻¹, which resulted in a strain rate of 1% m sec⁻¹. Visual examination of the fluorescent images of the networks subjected to the strain revealed qualitative changes in the networks; however these changes may be difficult to quantify by visual examination alone (FIG. 4).

To obtain a quantitative measure of the effect of the mechanical strain on the protein fibril network, confocal images of the network before and after stretch were obtained. Each image was divided into one pixel wide columns (FIG. 5). Pixel intensities for each column of the pre-stretch image, were compared or cross-correlated with pixel intensities of the corresponding column of the post-stretch image (FIG. 5). A perfect or nearly perfect match yields a cross-correlation value of 1, and the lack of a perfect or nearly perfect of a match yields a cross-correlation value less than 1. The cross-correlation value for each column was averaged over the entire image to obtain a net image cross-correlation value. A value of 1 for the net image cross-correlation value indicates that there were no changes to the network, and a value less than 1 indicates that the network geometry was changed as a result of the applied mechanical strain. The result of a network cross-correlation analysis performed as described above in a small region (100 μm²) is shown in FIG. 5 to the right. The results show that the method of cross-correlation of pixel intensities described in the foregoing is effective for quantification of changes in network structure as a function of applied mechanical strain. While fibronectin is not a primary protein of the ECM in the adult brain, it is present in the developing brain. It is therefore, important for understanding strain dependent architecture of the brain, and networks formed from fibronectin fibrils are suitable for the in vitro model of non-penetrating traumatic brain injury described herein.

The result of applying a stretching force on a fibronectin fibril network generated using the steps above is shown in FIG. 6. Network cross-correlation data was obtained by analyzing pixel intensities over a large area (500 μm²). The static confocal microscopy images (A, B) were captured before and after subjecting the network to a strain rate of 100% m sec⁻¹. The images were analyzed using custom MatLab script. Images were cross-correlated to elucidate changes in network structure (FIG. 6C). The results shows that the automated cross-correlation analysis described herein can provide a quantitative measure of structural change occurring in a protein network due to applied strain.

### Example 2

### Screening of Compounds for Treating or Preventing a Non-penetrating Traumatic Brain Injury

The in vitro model of a non-penetrating traumatic brain injury described in Example 1 may be used in a method for identifying a compound useful for preventing or treating a non-penetrating traumatic brain injury. The protein fibril network of the in vitro model is contacted with a test compound, and incubated for a period of time. A stretching force is next applied to the protein network. Confocal microscopy images of the network are acquired both before and after applying the stretching force. Changes in, for example, the geometry of the network are determined by computing cross-correlation of pixel intensities between the pre-stretch and the post-stretch images. A compound that is able to preserve the pre-stretch structure, e.g., geometry, of the protein network, or that results in only a minimal change to the pre-stretch structure, e.g., geometry, (i.e., a cross-correlation value of 1 or close to 1), is identified as a compound that is useful for preventing or treating a non-penetrating traumatic brain injury.

### Equivalents

In describing exemplary embodiments, specific terminology is used for the sake of clarity. For purposes of description, each specific term is intended to at least include all technical and functional equivalents that operate in a similar manner to accomplish a similar purpose. Additionally, in some instances where a particular exemplary embodiment includes a plurality of system elements or method steps, those elements or steps may be replaced with a single element or step. Likewise, a single element or step may be replaced with a plurality of elements or steps that serve the same purpose. Further, where parameters for various properties are specified herein for exemplary embodiments, those parameters may be adjusted up or down by 1/20th, 1/10th, ⅕th, ⅓rd, ½nd, and the like, or by rounded-off approximations thereof, unless otherwise specified. Moreover, while exemplary embodiments have been shown and described with references to particular embodiments thereof, those of ordinary skill in the art will understand that various substitutions and alterations in form and details may be made therein without departing from the scope of the invention.

1. A cell-free in vitro model of non-penetrating traumatic brain injury, comprising
a base layer comprising a stretchable membrane disposed on a surface, a network of extracellular matrix (ECM) protein fibrils on the stretchable membrane structured to mimic the brain perineuronal network,
wherein said network of ECM protein fibrils comprises a plurality of nodes, wherein each node comprises a polymer having a charge density of about 0.10 C/m² or greater, wherein each node is independently spaced from each of its immediate neighbors by about 20 μm to about 40 μm, wherein each fibril is attached to one or more of the plurality of nodes, and each fibril intersects another fibril at a node,
wherein the stretchable membrane comprising the plurality of nodes and the network of fibrils is stretched, thereby mimicking non-penetrating traumatic brain injury.

2. A cell-free in vitro model of non-penetrating traumatic brain injury, comprising
a base layer comprising a stretchable membrane disposed on a surface, a network of extracellular matrix (ECM) protein fibrils on the stretchable membrane structured to mimic the brain perineuronal network,
wherein said network of ECM protein fibrils comprises a plurality of nodes, wherein each node comprises a polymer having a charge density of about 0.10 C/m² or greater, wherein each node is independently spaced from each of its immediate neighbors by about 20 μm to about 40 μm, wherein each fibril is attached to one or more of the plurality of nodes, and each fibril intersects another fibril at a node,
wherein the stretchable membrane comprising the plurality of nodes and the network of fibrils when stretched, mimics non-penetrating traumatic brain injury.

3. The model of traumatic brain injury of claim 1 or claim 2, wherein each fibril is attached to at least one node and at least one of its nearest neighbor nodes.

4. The model of traumatic brain injury of claim 1 or claim 2, wherein the network of extracellular matrix proteins comprises a network of fibrils selected from the group consisting of aggrecan fibrils, brevican fibrils, neurocan fibrils, tenascin R fibrils, and any combination thereof.

5.-8. (canceled)

9. The model of traumatic brain injury of claim 1 or claim 2, wherein the base layer has an elasticity of about 0.5 megapascal (MPa) to about 1.5 megapascal (MPa) or about 0.75 megapascal (MPa) to about 1.25 megapascal (MPa).

10. (canceled)

11. (canceled)

12. The model of traumatic brain injury of claim 1 or claim 2, wherein the base layer comprises a silicone membrane.

13. The model of traumatic brain injury of claim 1 or claim 2, wherein the network of extracellular matrix proteins is stretched at a strain rate of about 0.1-10% msec-1 or about 0.5-5% msec-1.

14. The model of traumatic brain injury of claim 1 or claim 2, wherein the network of extracellular matrix proteins is stretched at a displacement rate of about 25 μmsec-1 to about 1000 μmsec-1 or about 100 μmsec-1 to about 500 μmsec-1.

15.-17. (canceled)

18. The model of traumatic brain injury of claim 1 or claim 2, wherein the polymer has a charge density of about 0.01 C/m² to about 10.0 C/m² or about 0.10 C/m2 to about 0.20 C/m2.

19. (canceled)

20. The model of traumatic brain injury of claim 1 or claim 2, wherein the polymer is a polystyrene sulfonate.

21. (canceled)

22. A method for identifying a compound useful for preventing or treating a non-penetrating traumatic brain injury, the method comprising
providing a cell-free in vitro model of traumatic brain injury of claim 2;
contacting said cell-free in vitro model with a test compound and examining the structure of the network of extracellular matrix (ECM) protein fibrils of the model;
generating a mechanical strain in the network of ECM protein fibrils previously determined to be sufficient to cause a structural change in the network;
examining the structure of the network after the generation of the mechanical strain for the presence of a change; and,
identifying the test compound as a compound useful for preventing or treating a traumatic brain injury if the change in the structure of the network after the generation of the strain is not substantial or is absent.

23. The method of claim 22, wherein the change is measured quantitatively.

24. The method of claim 22, wherein the change in the network is a change in the geometric structure of the network.
